SEQUENCE LISTING (11 sequences)

SEQ ID NO: 1 - protein, 1577 aa, Artificial Sequence
  zebrafish akap12 alpha form
MGATPSVQRDAKGPEDAPEDVSAPLSDAHDGETADGEPLQKNGQISISSLNGKTDDQTEYNGHTEENPPAEVGQTDTISPKEDSPETIEVHQEEVAPQMNGEKGDNSANADEITTAEEKVVEEKQEEANEVGFKKIFRFVGFKFTLKKDKNEKTEPVQLLTVKEAESGADAATEEKKEEPAAEEDRSVEEKSPETTENEAKAEEVTEKAEEPAEQTVVDAPSETEKVSDIETEKPAEETGTISEKEPEPEVPAESPTSPPSQETQSPFKRFFTQGIFSNLRKKASFKKPKDEEHVKEKPAEEDIKETEETAEGVPEATEEAKVDAENEPAEGEQIEKPSETVETKAETTTETTAETTNEVTPTEKEEQQDLKVEAEATSEVETVTHTEPAQAPAVETTQPTDDAKTSDKPDISEEAPIEPEILSSQEKSKAHGSPLKKLFTGAGLKKLSSKKHKNKKDAESKQTESSEQTAETVQSTESTEPQKPDSGASSPEESGEHVVGEVAQAEVAQAVEPDGDAVTSDGEKKKEGILPWSSFKKLVTPKKRVKRPSESEDEAPGDKPKFSTLSSTESAISDEKADEPKPSEEVPSKEELKEEAKEESQAESKTEPKAEKSESVAEEPKRKMDTSVSWEALICVGSSKKRARKTSDSDDEEAKIEEEVQPSEEEPIKTAESPLVSSGEADHENLASSPEPEGELVSTWESFKRLVTHRKKAKAEDKSDEASGPEQTTSDSETPKEESSFSLRKLIPRRKKKSDGKQEQVSSDVGSAEDDSDTPAVVPLSEYDSEPSAEAAVKAEEVKQESATVTQAKASAEDRSPSWISTTVENVEDETEGNQLSDIPEEGDTAATPKSTDNTIAEDIVELTSEAVTALEQVEETEMVSAVSRVTASPDTSGETTPVPGDGVERKTDVVIQEAVETISVTTNAMAVTMTEEQETVVAITTDALLVESATKEQKTVLVAHEKNEAVAVCTGLDTSEIRAVEEESLNQKPSVESATVVSQPLVTEVAVEEKTQEPERVTVTEDEVHEAQTSGVQAELKDQPIENAIEEKAQFEEIKDTPIAETVADIHEVAAVKVAVISAVQQEPEILEEPVMAEKSPEIESAGPVEATVEEAICAQTAEVTEFAVAEGEKVQELDDVKETVAAVEVASVENVSTAVTEEVMATLPEVPASQIAGSTEDPIPVVAATEEFAVIKETICVSSISETTESHSADIAKETLMENVPVVLSTGDHKMQVAVNEVEVVSAQGVVEGNIEAASTKLSVALEEVTENVKEETEVIQATQVTEAEIIEKQSSVIVQEIIQNVVENFAEAHGEQNVSEKTTEESCITSAEVKVNEISVETTEEGSSTDKLAASDNVCKDVEETRIVTEKPPKIVNEAIQIAETVPVSITDEIQAQNEEVASVWVADVHQETEVTKSEVELESDLKEVQAEIQQEIKAVPEEKTAETSVTVASQDQVVAEQCQVTLTAVQIETAQEFNVGVVNVINADDVPETNMKEDCSEGQESTEDRPQNDLEEPQTKVSTNQEESRGPDCQKTDAKNVPAKLEAASEMTSNATAAEEVGNEIEPVSTEVVTVS

SEQ ID NO: 2 - protein, 1533 aa, Artificial Sequence
  zebrafish akap12 beta form
MLGTITLTVGQTDTISPKEDSPETIEVHQEEVAPQMNGEKGDDSANADEITTAEEKVVEEKQEEANEVGFKKIFRFVGFKFTLKKDKNEKTEPVQLLTVKEAESGADAATEEKKEEPAAEEDRSVEEKSPETTENEAKAEEVTEKAEEPAEQTVVDAPSETEKVSDIETEKPAEETGTISEKEPEPEVPAESPTSPPSQETQSPFKRFFTQGIFSNLRKKASFKKPKDEEHVKEKPAEEDIKETEETAEGVPEATEEAKVDAENEPAEGEQIEKPSETVETKAETTTETTAETTNEVTPTEKEEQQDLKVEAEATSEVETVTHTEPAQAPAVETTQPTDDAKTSDKPDISEEAPTEPEILSSQEKSKAHGSPLKKLFTGAGLKKLSSKKHKNKKDAESKQTESSEQTAETVQSTESTEPQKPDSGASSPEESGEHVVGEVAQAEVAQAVEPDGDAVTSDGEKKKEGILPWSSFKKLVTPKKRVKRPSESEDEAPGDKPKSSTLSSTESAISDEKADEPKPSEEVPSKEELKEEAKEESQAESKTEPKAEKSESVAEEPKRKMDTSVSWEALICVGSSKKRARKTSDSDDEEAKIEEEVQPSEEEPIKTAESPLVSSGEADHENLASSPEPEGELVSTWESFKRLVTHRKKAKAEDKSDEASGPEQTTSDSETPKEESSFSLRKLIPRRKKKSNGKQEQVSSDVGSAEDDSDTPAVVPLSEYDSEPSAEAAVKAEEVKQESATVTQAKASAEDRSPSWISTTVENVEDETEGNQLSDIPEEGDTAATPKSTDNTIAEDIVELTSEAVTALEQVEETEMVSAVSRVTASPDTSGETTPVPGDGVERKTDVVIQEAVETISVTTNAMAVTMTEEQETVVAITTDALLVESATKEQKTVLVAHEKNEAVAVCTGLDTSEIRAVEEESLNQKPSVESATVVSQPLVTEVAVEEKTQEPERVTVTEDEVHEAQTSGVQAELKDQPIENAIEEKAQFEEIKDTPIAETVADIHEVAAVKVAVISAVQQEPEILEEPVMAEKSPEIESAGPVEATVEEAICAQTAEVTEVAVAEGEKVQELDDVKETVATVEVASVENVSTAVTEEVMATLPEVPASQIAGSTEDPIPVVAATEEFAVIKETICVSSISETTESHSADIAKETLMENVPVVLSTGDHKIQVAVNEVEVVAAQGVVEGNIEAASTKLSVALEEVTENVKEETEVIQATQVTEAEIIEKQSSVIVQEIIQNVVENFAEAHGEQNVFEKTTEESCITSAEVKVNEISVETTEEGSSSDKLAASDNVCKDVEETRIVTEKPPKIVNEAIQIAETVPVSITDEIKAQNEEVASVSVADVHQETEVTKSEVELKQAEEKSQSTKSEEGKSKVESDLKEVQAEIQQEIKAVPEEKTAETSVTVASQDQVVAEQCQVTLTAVQIETAQEFNVGVVNVINADDVPETNMKEDCSEGQESTEDRPQNDLEEPQTKVSTNQEDSRGPDCQKTDAKNVPAKLEAASEMTSNATAAEEVGNEIEPVSTEVVTVS

SEQ ID NO: 3 - DNA, 30 bp, Artificial Sequence
  forward primer of zebrafish akap12 alpha form
aaggatccatgggagcgacaccatccgtgc

SEQ ID NO: 4 - DNA, 26 bp, Artificial Sequence
  forward primer of zebrafish akap12 beta form
actttccaaagcagacaaccctcggg

SEQ ID NO: 5 - DNA, 36 bp, Artificial Sequence
  reverse primer of zebrafish akap12 alpha form
aagaattctcatgacactgtgacaacctctgtggag

SEQ ID NO: 6 - DNA, 35 bp, Artificial Sequence
  reverse primer of zebrafish akap12 beta form
agacatgattttgtatccatactattaacagcttg

SEQ ID NO: 7 - DNA, 25 bp, Artificial Sequence
  morpholino of zebrafish akap12 alpha form
tcttacctgttagagttattgtccc

SEQ ID NO: 8 - DNA, 25 bp, Artificial Sequence
  morpholino of zebrafish akap12 beta form
taccttgccatctgcggtttctcca

SEQ ID NO: 9 - DNA, 30 bp, Artificial Sequence
  forward primer for riboprobe
gaagaatctggtgaacatgttgtaggggaa

SEQ ID NO: 10 - DNA, 26 bp, Artificial Sequence
  reverse primer for riboprobe
gcgacaacctcaacctcattcactgc

SEQ ID NO: 11 - DNA, 2258 bp, Artificial Sequence
  AKAP12 riboprobe
gaagaatctggtgaacatgttgtaggggaagtagctcaagctgaggtggcccaagcagtagagcctgatggtgatgcagtcacttctgatggtgaaaagaagaaagaaggaattttaccttggtcctctttcaaaaaactagtcactccaaaaaaacgtgtcaaaaggccctctgagagtgaagatgaagcacctggagacaaacccaaattttctaccctatcttcaacagagagtgccatctctgatgagaaagctgatgaacctaaaccatcagaggaggtaccatctaaagaagagcttaaagaagaggctaaagaagagtcacaggcagaatctaagacagaaccaaaagctgagaagtctgagtctgttgcagaagagccaaagagaaaaatggatacatctgtgtcctgggaagctcttatttgtgtggggtcatctaaaaaaagggccagaaagacttctgattctgatgatgaagaagctaaaattgaagaagaagtgcagccatccgaagaagagccgataaagactgctgagtcccctcttgtaagctctggtgaagctgaccatgagaatttagcttcttcccctgaaccagagggagaacttgtttctacctgggagtcctttaagaggctggtaacccacagaaagaaagctaaagcagaagacaaatctgatgaagcctcaggcccagagcagacaacctctgacagtgaaaccccaaaagaagagtcctctttctctttgagaaaactgattccacgcaggaagaagaagtctgatggtaagcaagagcaggtatcgtctgacgtcggctctgcagaggatgactctgatactccagctgtggtgcctctttcagaatatgacagcgaaccatctgcagaagctgcagttaaggcagaggaggtaaagcaggagtctgcaacagtcactcaggcaaaggcctcagctgaagatcgatcaccatcttggatctcaaccactgtagaaaatgttgaggatgaaacagagggaaaccaattaagtgatatacctgaagaaggcgacacagctgccacacctaaatccactgacaacaccattgcagaggacattgtggagcttacatcagaagctgttacagcccttgaacaagtagaagagactgaaatggtttctgctgtttcacgtgttacagcatcaccagatacatcaggcgagacaacccctgtccctggtgatggtgtagagaggaagactgatgtagttattcaagaagctgtggaaactatcagtgttaccacaaatgccatggctgtcactatgacagaggaacaggaaacagtagttgccatcaccactgatgctcttctggttgagtctgccaccaaagaacagaaaacagtcttggtggcgcatgagaaaaacgaggcagtggccgtttgcactggccttgacactagtgagataagggcagtggaggaagaaagcctcaatcagaagccttcagtagagtcagccactgttgttagccagccactagtcactgaggtggctgttgaagaaaaaacacaggagcctgaaagggttactgtgactgaggatgaggtccatgaggctcagactagcggagttcaggcagagctaaaagatcagcccatagaaaatgcaattgaagagaaagcgcagtttgaggagattaaagacactcctatagcagaaactgtggctgatatccatgaagtagcagcagttaaagtagcggtgatcagtgctgtgcaacaggaaccagaaatccttgaagagccagtgatggcagaaaagtcccctgagattgagtctgcaggtcctgttgaagcaactgttgaagaggcaatctgtgctcaaactgcagaagtcactgaatttgctgttgctgaaggtgagaaagtgcaagaacttgatgatgttaaagagactgttgccgcagttgaagttgcatctgtagagaatgtttccacagccgttaccgaggaggtcatggcaacccttccagaggtacctgcttctcaaattgctggatctacagaagatcccattcctgttgtcgctgccactgaagaatttgcagtcatcaaagagaccatctgtgttagctcaatatctgagacaacagaatcccattcagcagatatagctaaagaaactcttatggaaaatgttcctgttgttctttccacaggtgaccacaaaatgcaagttgcagtgaatgaggttgaggttgtctc

(19) United States
(12) Patent Application Publication
(10) Pub. No.: US 2011/0162092 A1
(43) Pub. Date: Jun. 30, 2011

(54) COMPOSITION COMPRISED OF AKAP12 AND USES OF AKAP12 MUTANT ZEBRAFISH AS AN ANIMAL MODEL

(75) Inventors: Kyu-Won Kim, Seoul (KR); Hyouk-Bum Kwon, Seoul (KR)

(73) Assignee: SNU RDB FOUNDATION, Seoul (KR)

(21) Appl. No.: 12/997,653

(22) PCT Filed: Jun. 12, 2009
(86) PCT No.: PCT/KR09/03178

§ 371 Date: Mar. 16, 2011

(30) Foreign Application Priority Data
Jun. 13, 2008 (KR) 10-2008-0055700

(51) Int. Cl. A61K 38/45; C12N 9/12; G01N 33/00; A61P 7/04

(52) U.S. Cl. 800/3; 435/194; 424/94.5

(57) ABSTRACT

The present invention relates to a composition comprised of AKAP12 (A-Kinase anchoring protein 12) and to uses of AKAP12 mutant zebrafish as an animal model. More particularly, the following characteristics are noted in the present AKAP12 mRNA knockdown zebrafish: crooked or shortened tail, inability to move normally, non-uniform micro-vasculature in the brain, and change in heart shape with non-uniform and weak heartbeats. It also has various circulatory and genetic defects, such as hemorrhage from the ventricles of the heart, brain, and retina. All of these defects can be cured with AKAP12 injection. Therefore, AKAP12 can be used as an active component for a composition to prevent and heal circulatory and genetic defects that are caused by AKAP12 deficiency, and as a hemorrhage inhibitor. Further, the AKAP12 deficient mutant zebrafish can be useful as an animal model for verification of effectiveness of treatment for genetic defects in the circulatory system.

### TECHNICAL FIELD

The present invention relates to a composition comprised of A-Kinase anchoring protein 12 (AKAP12) as an active component, for prevention and treatment of defects of a circulatory system including vessels and hearts induced by the deficiency of AKAP12, composition for prevention and treatment of embryological defect, bleeding inhibitor comprised of AKAP12 as an active component, and use of AKAP12-deficient mutant zebrafish as an animal model to verify the effectiveness of a medicine for treatment of circulatory or genetic defects.

### BACKGROUND ART

The zebrafish (zebra danio, Danio rerio) is tropical fish used widely as a model for scientific researches. The zebrafish can replace animal models such as mice particularly in the researches of development and gene functions of vertebrate, and is advantageous in terms of relatively shorter time for development, larger and stronger embryos, and transparent embryos that allow better observation. The zebrafish can be utilized in a specific gene function research by reducing an amount of gene expression through gene splicing of RNA to a specific gene using morpholino antisense technology and gene knockdown (Froese, R., et al., Danio rerio. FishBase. Retrieved on 2007-04-07). However, no case has been reported as of yet regarding the use of AKAP12 mutant zebrafish as an animal model to verify the effectiveness of a circulatory defect medicine.

Morpholino is a molecule that regulates gene expression and widely used as a reagent to knock down gene expression by blocking translation and gene splicing of the RNA by binding to less than 25 base pairs of the RNA (Nasevicius, A et al., Nature Genetics 26 (2): 216-220, 2000).

AKAP12 (A-Kinase anchoring protein 12) (Gravin, SSeCKS (Src-suppressed C kinase substrate)) is one of scaffolding proteins existing within a cell, and is the first protein that is known to be reactive with oncogene such as src or ras to be down-regulated (Frankfort B J, et al., Biochem Biophys Res Commun, January 26; 206(3): 916-26, 1995). The AKAP12 is known to be particularly reactive with the beta-adrenergic receptor, PKC, PKA, phosphodiesterase, Calmodulin and F-actin to influence signal transmission (Wang H Y, et al., Eur J. Cell Biol., July; 85(7): 643-50, 2006), and also reported for its influence on the cell migration, mitosis, blood barrier and apoptosis (Weiser D C, et al., GENES & DEVELOPMENT 21: 1559-1571, 2007; Xia W, Experimental Cell Research, Volume 277, Issue 2, p 139-151, 2002; Choi Y K, et al., The Journal of Neuroscience, April 18, 27(16):4472-4481, 2007; Lee S W; et al., Nature Medicine Article, 1 July, 2003; Yoon D K, et al., Cancer Letters, Volume 254, Issue 1, Pages 111-118, 2007). Although there are some reports about the link of AKAP12 with the development, i.e., with the overall defect pattern of the initial embryonic structure (Weiser D C, et al., Genes Dev., June 15; 21(12): 1559-71, 2007), no case has been reported yet regarding a link between defects of the respective sub-organs such as bleeding due to heart defect, cerebral vascular stability defect or vascular defect of the zebrafish with the two zebrafish AKAP12 isoforms (alpha and beta).

Therefore, by injecting morpholino into zebrafish, knocking down AKAP12 mRNA of zebrafish, and observing the phenotype, the present inventors confirmed that more circulatory and genetic defects appeared than in normal zebrafish and thus completed the present invention based on the confirmed function of AKAP12.

### DISCLOSURE

### Technical Problem

An object of the present invention is to provide a composition comprised of AKAP12 (A-Kinase anchoring protein 12) as an active component; for prevention and treatment of circulatory and genetic defect induced due to AKAP12 deficiency, a composition comprised of AKAP12 as an active component for bleeding inhibition, and an AKAP12-deficient mutant animal with circulatory and genetic defects.

### Technical Solution

In order to accomplish the above-mentioned object, the present invention provides a composition comprised of AKAP12 (A-Kinase anchoring protein 12) as an active component, for prevention and treatment of circulatory defect induced due to AKAP12 deficiency.

Further, the present invention provides a composition comprised of AKAP12 as an active component, for prevention and treatment of genetic defect induced due to AKAP12 deficiency.

Further, the present invention provides a composition comprised of AKAP12 as an active component for bleeding inhibition.

Further, the present invention provides a method of treatment or prevention of circulatory defect, comprising a step of administering a pharmaceutically-effective amount of AKAP12 into a subject.

Further, the present invention provides a method of treatment or prevention of genetic defect, comprising a step of administering a pharmaceutically-effective amount of AKAP12 into a subject.

Further, the present invention provides a use of AKAP12 for a preparation of a composition for prevention and treatment of circulatory defect.

Further, the present invention provides a use of AKAP12 for a preparation of a composition for prevention and treatment of genetic defect.

Further, the present invention provides a use of AKAP12 for a preparation of a composition for bleeding inhibition.

Further, the present invention provides an AKAP12-deficient mutant animal with a circulatory defect.

Further, the present invention provides an AKAP12-deficient mutant animal with a genetic defect.

Further, the present invention provides a method for preventing circulatory defect and a method for screening a medicine, comprising the steps of:

1) administering a candidate for prevention and treatment of a circulatory defect into an AKAP12 gene knockout or knockdown animal;

2) confirming a degree of circulatory development, of the animal into which the candidate for prevention and treatment of circulatory defect is administered in step 1); and

3) selecting a candidate which meaningfully recovers the degree of the circulatory development by comparing with a control group animal into which the candidate is not administered.

Further, the present invention provides a method for preventing genetic defect and a method for screening a medicine, comprising the steps of:

1) administering a candidate for preventing and treating a genetic defect into an AKAP12 gene knockout or knockdown animal;

2) confirming a degree of genetic development of the animal into which the candidate for preventing and treating the genetic defect is administered in step 1); and

3) selecting a candidate which meaningfully recovers the degree of genetic development by comparing with a control group animal into which the candidate is not administered.

The terminology used throughout the disclosure will be explained below.

The term “knockout” herein refers to inducing of a complete deactivation in a normal gene by externally introducing DNA with defective gene.

The term “knockdown” herein refers to reducing of an amount of gene expression by degrading mRNA with RNAi or the like.

The term “prevent” herein refers to all the behaviors that delay the circulatory or genetic defect by administering a composition according to the present invention.

The term “treat” and “recover” herein refer to all the behaviors that improve or change the symptom of the circulatory or genetic defect to better state by administering a composition according to the present invention.

The term “administer” herein refers to providing a subject with a predetermined composition according to the present invention by a predetermined appropriate method.

The term, “subject” herein refers to all the animals including human, monkeys, dogs, goats, pigs or mice, etc., that can have improved symptoms of circulatory or genetic defect by administration of a composition according to the present invention.

The expression “pharmaceutically-effective amount” refers to an amount sufficient to treat the disease, which is reasonable benefit or risk ratio applicable for medical treatment, and this can be determined according to the factors well known in the medical science, including types and severity of atopy, activity of drug, sensitivity to drug, time period for administration, path of administration and discharge rate thereof, treatment period, factors including concurrently-used drugs, and the like.

The present invention will be explained in greater detail below.

The present invention provides a composition comprised of A-Kinase anchoring protein 12 (‘AKAP12’) as an active component, for prevention and treatment of circulatory detect induced due to AKAP12 deficiency.

The present invention also provides a composition comprised of AkAP12 as an active component for prevention and treatment of genetic defect induced due to AKAP12 deficiency.

The AKAP12 may preferably be an AKAP12 alpha form with a nucleic sequence represented by SEQ. ID. No. 1, or an AKAP12 beta form with a nucleic sequence represented by SEQ. ID. No. 2, but not limited thereto.

The present inventors cloned the zebrafish AKAP12 alpha form and the zebrafish AKAP12 beta form, and prepared morpholino to knock down mRNA of the zebrafish AKAP12 alpha form and the zebrafish. AkAP12 beta form. The prepared morpholino attaches to a site where the characteristic: variant regions of the alpha and beta forms are spliced to thus prevent the splicing and development into a mature mRNA.

In order to check the mRNA expression pattern of the zebrafish AKAP12, the inventors prepared riboprobe corresponding to the base sequence commonly included in two isoforms of the AkAP12 alpha and beta forms, and carried out in situ hybridization (ISH). As a result, the AKAP12 generally expressed to blastoderm until 24 hours after the fertilization, but the expression was limited to head and large veins (dorsal aorta, DA), posterior cardinal vein (PCV) and intersegmental vessels (ISV) after 24 hours (see FIG. 2).

According to the present invention, the AKAP12 can cause normal development of circulatory defect of microvasculature in brain, heart and the entire veins, and also can cause normal development of shape and mobility, but not limited thereto.

In order to investigate influence of AKAP12 on the development of shape, the present inventors injected morpholino into zebrafish embryo, thereby knocking down AKAP12 alpha form and beta forms, and then observed the shapes. As a result, normal zebrafish in which AKAP12 mRNA is not knocked down; had straightforward and long tail portion and showed normal mobility. However, the zebrafish in which AKAP12 mRNA alpha and beta forms are knocked down showed crooked or, shortened tail portions and also unable to move normally.

The inventors injected morpholino for zebrafish AKAP12 alpha form into zebrafish embryos and observed the pattern of mRNA expression of AkAP12 alpha form in accordance with the degrees of defects in the AKAP12 alpha form knockdown zebrafish by RT-PCR. As a result, it was observed that severer defects were linked to a lower degree of mRNA expression (see FIG. 3).

The inventors injected morpholino for zebrafish AKAP12 alpha and beta forms sequentially in the order of concentration, and as a result, could confirm that the beta form showed the above-mentioned genetic defect at concentration above 7.5 nm and alpha form showed the genetic defect at 3.7 ng, respectively (see FIG. 4).

The above result shows that the AKAP12 develops the shape and mobility developmentally.

In order to confirm the influence of AKAP12 on the development of the micro-vasculatures, the present inventors knocked down AKAP12 alpha forms in the zebrafish and observed the pattern of circulatory defects of micro-vasculatures in brains. As a result, AKAP12 alpha knockdown zebrafish did not show the uniform micro-vascular pattern of the brain of the normal zebrafish, and fluorescence moved out of the micro-vasculatures and distributed therearound (see FIG. 5).

In order to confirm the influence of AKAP12 on the vascular development, the present inventors knocked down AKAP12 alpha and beta forms, respectively, using transgenic zebrafish, and observed defect patterns in the vessels. As a result, the AKAP12 alpha and beta knockdown, zebrafish did not show uniform vascular shapes of the normal zebrafish, and fluorescence moved out of the vessels and distributed therearound (see FIG. 6). It was additionally observed that the vein endothelial cells of the AKAP12 alpha and beta knockdown zebrafish had loose contacts among cells and also active movement (see FIG. 7).

In order to investigate the relationship between the active movement of the vein endothelial cells of the AKAP12 knockdown zebrafish with RhoA, which is one of the small GTPase proteins, the present inventors treated the AKAP12 knockdown zebrafish with ROCKOUT which is the RhoA signal inhibitor, and as a result, could confirm that the excessive movement of the vein endothelial cells to decrease (see FIG. 8).

The present inventors could also confirm the increase of Rho-GTP which is the active form of RhoA in the AKAP12 knockdown HUVEC, based on the in vitro analysis of RhoA-GTP binding assay using Human Umbilical Vein Endothelial Cells (see FIG. 9). Further, as a result of performing in vitro permeability assay using HUVEC, the inventors could confirm that AKAP12ab group treated with AKAP12 siRNA showed increased permeability of RITC compared to a control siRNA (sc), and decreased permeability when treated with ROCKOUT (see FIG. 10).

In order to determine if the loosening of contact among the vein endothelial cells in the AKAP12 knockdown zebrafish is due to deficiency of cell-cell adhesion protein, when AKAP12 genes are knocked down with siRNA, the inventors observed abnormality of ve-cadherin, which is one of adhesion proteins in cell membrane, in its expression and membrane-localization on the cell membrane, by western-blotting and immunocytochemistry, and also confirmed that the abnormality had recovered when AKAP12 was treated with both siRNA and ROCKOUT (see FIG. 11).

Accordingly, it was confirmed that AKAP12 is involved with the forming of veins and micro-vasculatures in brains, and that the zebrafish AKAP12 could influence the maturity of the veins and micro-vasculatures in brains.

In order to investigate the influence of AKAP12 on the development of hearts, the present inventors observed knocked down AKAP12 alpha form in the transgenic zebrafish which selectively expresses fluorescence in the light chain of the heart muscle and then observed the circulatory defect pattern of the hearts. As a result, the AKAP12 alpha knockdown zebrafish did not show uniform shape and arrangement of atriums and ventricles of the normal zebrafish, non-uniform and weak heartbeats, and in the case of severe defect, obstructed bipod circulation (see FIG. 12).

Accordingly, it was confirmed that AKAP12 influences the forming of the heat heavily.

Further, the preset invention provides a composition comprised of AKAP12 as an active component for hemorrhage inhibition.

The AKAP12 may preferably be an AKAP12 alpha form with a nucleic sequence represented by SEQ. ID. No. 1, or an AKAP12 beta form with a nucleic sequence represented by SEQ. ID. No. 2, but not limited thereto.

In order to verify the influence of AKAP12 on the hemorrhage, the present inventors knocked down AKAP12 alpha and beta forms and observed zebrafish embryos. As a result, the inventors could observe bleeding in the zebrafish at days 2 and 3. The bleeding was observed generally from the ventricles in brains, retina, hearts and trunks, and it was statistically indicated that as the amount of morpholino for zebrafish AKAP12 alpha and beta forms increased, the rate of zebrafish with hemorrhage increased (see FIGS. 13, 14 and 15).

In order to verify the changes in zebrafish veins with hemorrhage, the present inventors observed vasculature pattern in brain using transgenic zebrafish. As a result, a group without hemorrhage formed vessels, although it showed non-uniform pattern of curves in the vessel pattern of the brains, which is different from the normal zebrafish vessels of the brains into which morpholino had not been injected. On the other hand, a group with hemorrhage formed thinned vessels in brains, along with the non-uniform vessel patterns in brains (see FIG. 16).

The present inventors also observed genetic defect and circulatory defect patterns in hearts and vessels, using amounts of morpholino for zebrafish AKAP12 alpha and beta forms. As a result, the inventors could confirm that the zebrafish AKAP12 alpha form had bleeding with the treatment of morpholino in an amount of 1 ng 2 ng, concurrent bleeding and vein defects in hearts and trunks with morpholino in an amount of 3 ng 4 ng, and defects in hearts and other veins which were severe enough to block efficient blood circulation, resulting in a reduction in the amount of bleeding with the treatment of morpholino at an amount of 4 ng or more. Further, the zebrafish AKAP12 beta form showed concurrent bleeding and vein defects in hearts and trunks when treated with morpholino in an amount of 7.5 ng 8 ng, and only the vein defects in hearts and trunks when treated with morpholino in an amount of 9 ng or more (see FIG. 17).

Accordingly, it was confirmed that AKAP12 causes the circulatory systems including hearts and veins to develop and thus provides hemorrhage inhibition effect.

In order to confirm if the genetic defects observed as explained above are linked to AKAP12-specific knockdown, the present inventors conducted rescue experiment using zebrafish AKAP12 alpha and beta mRNA and morpholino to thus confirm if the defects in the shapes and hemorrhage were due to AKAP12-specific knockdown. As a result, the inventors could confirm that a control group into which only the morpholino for AKAP12 alpha and beta forms was microinjected showed a defect pattern of crooked trunks and bleeding, while a test group into which a mixture of the zebrafish AKAP12 alpha and beta mRNA and morpholino was injected showed increasing rate of zebrafish with improved tail and heart defects, as the amount of mRNA increased (see FIG. 19).

In consideration of the above test results, the AKAP12 according to the present invention inhibits or relieves the defects in shapes and genetic defects such as mobility defect, inhibits circulatory defects including heart and vein defects, and inhibits hemorrhage. Accordingly, the AKAP12 according to the present invention can be used as an active component for prevention and treatment of circulatory defects induced due to AKAP12 deficiency, prevention and treatment of genetic defects, and hemorrhage inhibition.

The composition according to the present invention may include at east one type of active component having the same or similar function in addition to AKAP12. For administration, at least one type of pharmaceutically-acceptable carrier may be additionally used.

The composition comprised of the AKAP12 may be administered parenterally for clinical administration, and used in the form of a general pharmaceutical preparation. That is, the composition comprised of AKAP12 according to the present invention, may be prepared into preparations using generally-used diluting agent or excipient including filler, carrier, binder, wetting agent, disintegrator, or surfactant.

The pharmaceutically-acceptable carrier may be saline solution, sterile water, Ringer solution, buffer saline solution, dextrose solution, maltodextrin solution, glycerol, ethanol and a mixture of at least one from among the above, and if necessary, other general additives such as oxidant, buffer solution, or fungistats may be added. Further, diluents, dispersant, surfactant, binder and lubricant may be additionally added to provide a dosage form using aqueous solution, suspension, and emulsion.

The composition according to the present invention may be administered by a well-known methods including intra arterial injection, intravenous injection, percutaneous injection, intranasal administration, transbronchial administration, or intramuscular administration.

A dose of the composition of the present invention may vary depending on the weight, age, gender, heath condition, diet, time of administration, method of administration, excretion rate, and severity of disease. For example, a daily dose may preferably be 0.01˜5000 mg/kg, and more preferably 0.01˜10 mg/kg, but not limited thereto. It is also preferable to administer the composition from one to several times a day.

Further, the present invention provides a method for treatment of genetic defect, including a step of administering a pharmaceutically-effective amount of AKAP12 into a subject with a circulatory defect induced due to AKAP12 deficiency.

Further, the present invention provides a method for prevention of a circulatory defect, including a step of administering a pharmaceutically-effective amount of AKAP12 into a subject.

Further, the present invention provides a method for treatment of a genetic defect, including a step of administering a pharmaceutically-effective amount of AKAP12 into a subject with a genetic defect induced due to AKAP12 deficiency.

Further, the present invention provides a method for prevention of a genetic defect, including a step of administering a pharmaceutically-effective amount of AKAP12 into a subject.

The AKAP12 according to the present invention inhibits or relieves the defects in shapes and genetic defects such as mobility defect, inhibits circulatory defects including heart and vein defects, and inhibits hemorrhage. Accordingly, the AKAP12 according to the present invention can be used as an active component for prevention and treatment of circulatory defects induced due to AkAP12 deficiency, prevention and treatment of genetic defects, and hemorrhage inhibition.

The AKAP12 may preferably be an AKAP12 alpha form with a nucleic sequence represented by SEQ. ID. No. 1, or an AKAP12 beta form with a nucleic sequence represented by SEQ. ID. No. 2, but not limited thereto.

The composition according to the present invention may include at east one type of active component having the same or similar function in addition to AKAP12. For administration, at least one type of pharmaceutically-acceptable carrier may be additionally used.

The composition comprised of the AKAP12 may be administered parenterally for clinical administration, and used in the form of a general pharmaceutical preparation. That is, the composition comprised of AKAP12 according to the present invention may be formed into preparations using generally-used diluting agent or excipient including filler, carrier, binder, wetting agent, disintegrator, or surfactant.

The parenternal administration may include a well-known methods including intra arterial injection, intravenous injection, percutaneous injection, intranasal administration, transbronchial administration, or intramuscular administration.

A dose of the composition of the present invention may vary depending on the weight, age, gender, heath condition, diet, time of administration, method of administration, excretion rate, and severity of disease. For example, a daily dose may preferably be 0.01˜5000 mg/kg, and more preferably 0.01˜10 mg/kg, but hot limited thereto. It is also preferable to administer the composition from one to several times a day.

Further, the present invention provides a use of AKAP12 for a preparation of a composition for prevention and treatment of a circulatory defect.

Further, the present invention provides a use of AKAP12 for a preparation of a composition for prevention and treatment of a genetic defect.

Further, the present invention provides a use of AKAP12 for a preparation of a composition for hemorrhage inhibition.

The present invention inhibits or relieves a defect in appearance and a genetic defect such as mobility defect, inhibits a circulatory defect including heart and vein defects, and inhibits hemorrhage. Accordingly, the AKAP12 according to the present invention can be used as an active component for prevention and treatment of circulatory defects induced due to AkAP12 deficiency, prevention and treatment, of genetic defects, and hemorrhage inhibition.

The AKAP may be an AKAP12 alpha form with a nucleic sequence represented by SEQ. ID. No. 1, or an AKAP12 beta form, with a nucleic sequence represented by SEQ. ID. No. 2, but not limited thereto.

Further, the present invention provides an AKAP12-deficient mutant animal having a circulatory defect.

Further, the present invention provides an AKAP12-deficient mutant animal having a genetic defect.

The AKAP may be AKAP12 alpha form with a nucleic sequence represented by SEQ. ID. No. 1, or an AKAP12 beta form with a nucleic sequence represented by SEQ. ID. No. 2, but not limited thereto.

The AKAP12-deficient mutant may be AKAP12 gene knockout or knockdown, but not limited thereto.

The animal may be one selected from the group consisting of zebrafish, mouse, rat, pig and monkey, and more preferably zebrafish, but not limited thereto.

Further, the present invention provides a method for screening a medicine for prevention and treatment of a circulatory defect, including steps of:

1) administering a candidate for prevention and treatment of a circulatory defect into an AKAP12 gene knockout or knockdown animal;

2) confirming a degree of circulatory development of the animal into which the candidate for prevention and treatment of the circulatory disease is administered in step 1); and

3) selecting a candidate which meaningfully recovers the degree of circulatory development by comparing with a control group animal into which the candidate is not administered.

Further, the present invention provides a method for screening a medicine for prevention and treatment of a development defect, including steps of:

1) administering a candidate for prevention and treatment of the genetic defect into an AKAP12 gene knockout or knockdown animal;

2) confirming a degree of genetic development of an animal into which the candidate of the medicine for prevention and treatment of the genetic defect is administered in step 1); and

3) selecting a candidate which meaningfully recovers the degree of genetic development by comparing with a control group into which the candidate is not administered.

In the above methods, in step 1), the animal is preferably zebrafish, mouse, rat, pig or monkey, and more preferably, zebrafish, but not limited thereto.

In the above methods, the candidate in step 1) is preferably one selected from the group consisting of peptide, protein, non-peptide compound, synthetic compound, fermented product, cell extract, plant extract, animal tissue extract, and plasma, but not limited thereto. The compounds may be novel or known compounds.

The candidate may form a salt. The salt of the candidate may be physiologically-acceptable acid (e.g., inorganic acid) or base (e.g., organic acid), or preferably, physiologically-acceptable acidified salt. For example, salt of inorganic acid (e.g., hydrochloric acid, phosphoric acid, hydrobromic acid, or sulfuric acid), or salt of organic acid (e.g., acetic acid, formic acid, propionic acid, fumaric acid, maleic acid, succinate, tartaric acid, citric acid, malic acid, oxalic acid, benzoic acid, methane sulfonic acid, or benzene sulfonic acid) may be used.

The method for administering the candidate may be a parenteral administration selected from among, for example, intravenous injection, hypodermic administration, intracutaneous administration, and intraperitoneal administration, according to symptom of a subject animal and property of the candidate. Further, the dose of the candidate may be selected appropriately according to the administration method, property of the candidate, or the like.

### ADVANTAGEOUS EFFECTS

The AKAP12 (A-Kinase anchoring protein 12) according to the present invention has the function of recovering a circulatory or genetic defect induced due to AKAP12 deficiency in shapes, mobility, micro-vasculatures in brains, or hearts, and inhibiting hemorrhage, and accordingly, a composition comprised of AKAP12 may be used as a medicine for prevention and treatment of a circulatory defect, a medicine for prevention and treatment of a genetic defect, and a hemorrhage inhibitor. Further, since an AKAP12-deficient mutant zebrafish exhibits the circulatory or development defect mentioned above, the AKAP12-deficient mutant zebrafish can be effectively used as an animal model for screening a medicine for prevention and treatment of the circulatory or genetic defect.

### BRIEF DESCRIPTION OF DRAWINGS

FIG. 1 illustrates a sequence in which morpholino binds to the zebrafish AKAP12 alpha form mRNA and, zebrafish AKAP12 beta form mRNA (<->:morpholino binding site).

FIG. 2 illustrates an expression pattern of morpholino of zebrafish AKAP12, observed after preparing riboprobe, which is the base sequence included commonly in the zebrafish AAP12 alpha and beta forms, and performing in situ hybridization (hpf: hour post-fertilization).

FIG. 3 illustrates a result of observing AKAP12 alpha mRNA expression pattern according to respective degrees of defects (N, D1, D2, D3) among the zebrafish AKAP12 alpha form knockdown zebrafish by RT-PCR.

FIG. 4 illustrates a result of observing a minimum amount of genetic defect after injecting amounts of morpholino for AKAP12 alpha and beta forms in sequence.

FIG. 5 illustrates a result of observing through confocal microscope after injecting red fluorescent lysine-fixable tetramethylrhodamine-dextran through common cardinal the vein of AKAP12 alpha form knockdown zebrafish, in which ‘uninj (uninjection)’ refers to a zebrafish group into which morpholino for zebrafish AKAP12 alpha form has riot been microinjected.

FIG. 6 illustrates a result of observing through, confocal microscope the common cardinal vein of AKAP12 alpha and beta form knockdown zebrafish using lysine-fixable tetramethylrhodamine-dextran, in which red is lysine-fixable tetramethylrhodamine-dextran dye, and green indicates endothelial cells.

FIG. 7 illustrates a result of observing through confocal microscope the endothelial cells of the AKAP12 alpha and beta form knockdown zebrafish using lysine-fixable tetramethylrhodamine-dextran.

FIG. 8 illustrates a result of observing through confocal microscope the endothelial cells using lysine-fixable tetramethylrhodamine-dextran, after treating the AKAP12 alpha and beta form knockdown zebrafish with ROCKOUT which is a RhoA signal inhibitor.

FIG. 9 illustrates the level of GTP-RhoA as a result of RhoA-GTP binding assay, after knocking down AKAP12 by treating HUVEC (Human Umbilical Vein Endothelial Cells) with AKAP12 siRNA, in which
sc refers to siRNA control group, and
AKAP12ab refers to AKAP12 siRNA group.

FIG. 10 illustrates the level of permeability as a result of permeability assay, after knocking down AKAP12 by treating HUVEC with AKAP12 siRNA, in which
AKAP12ab refers to a group treated with AKAP12 siRNA, and
sc refers to a siRNA control group.

FIG. 11 illustrates the level of expression of ve-cadherin which is a cell membrane adhesion protein, using western-blotting and immunocytochemistry, after knocking down AKAP12 by treating HUVEC with AAP12 siRNA.

FIG. 12 illustrates a result of observing through confocal microscope the heart of the AKAP12 alpha form knockdown zebrafish.

FIG. 13 illustrates the site of hemorrhage shown in zebrafish embryos 2 days after, knocking down the zebrafish AKAP12 alpha and beta forms.

FIG. 14 illustrates a site of hemorrhage shown in zebrafish embryos 3 days after knocking down the zebrafish AKAP12 alpha and beta forms.

FIG. 15 is a graphical representation of a rate of zebrafish having hemorrhage in 2 and 3 days according to amounts of morpholino for zebrafish AKAP12 and AKAP12 beta forms.

FIG. 16 illustrates a result of observing through confocal microscope the vessels in brain after microinjecting morpholino for zebrafish AKAP12 alpha form into transgenic (fli:egfp) zebrafish embryos, in which
A denotes a control group,
B to D denote AKAP12 morphants (3 ng),
B denotes morphant without hemorrhage, and
C and D denote morphants with hemorrhage in ventricles.

FIG. 17 illustrates a result of observing a defect pattern according to amounts of morpholino, when the zebrafish AKAP12 alpha and beta form mRNA are knockdown.

FIG. 18 illustrates a degree of recovery of the defects in trunks and hemorrhage pattern after injecting morpholino for zebrafish AKAP12 alpha and beta form into zebrafish embryos.

FIG. 19 illustrates a result of observing defects after injecting morpholino for zebrafish AKAP12 alpha form, and a mixture of said morpholino for zebrafish AKAP12 alpha form and rat AKAP12 alpha form mRNA into zebrafish embryos.

### BEST MODE

### Mode for Invention

Hereinbelow, the present invention will be explained in greater detail below with reference to embodiments and examples.

However, the examples and experiments explained below are only written for illustrative purpose and should hot be construed as limiting the invention.

### Example 1

### Rearing of Zebrafish and Preparation of Zebrafish Embryos

Wild type zebrafish was purchased from Seijin Aquarium (South Korea), and transgenic zebrafish was purchased from, ZFIN website of the University of Oregon. The zebrafish was reared under condition (temperature: 28° C., light and shade: light on from 9:00 am to 9:00 pm, and off at other times, feed: brine shrimps). To obtain embryos, a partition was used to separate female and male zebrafish from each other one night before mating, and the partition was removed the next day for mating with turning on light. The zebrafish eggs from the mating were moved to an agar gel frame.

### Example 2

### Cloning Zebrafish AKAP12 Alpha and Beta Form Genes

In order to study the zebrafish AKAP12, the prevent inventors cloned zebrafish AKAP12 alpha and beta forms. The inventors searched possible gene sequences of the zebrafish AKAP12 alpha form (gene code No.: xm_{—}690658.2) and AKAP12 beta form ((gene code No.: ef539208) on the website of the Korean National Center for Biotechnology Information (www.ncbi.nlm.nih.gov) and aligned the obtained gene sequences with the sequence of the zebrafish chromosome 20 (gene code No.: cr926887), and as a result, could confirm that CDS; in the form of zebrafish AKAP12 alpha and beta forms exist in the zebrafish chromosome 20. For the purpose of cloning, the inventors prepared forward primer alpha form: AAGGATCCATGGGAQCGACACCATCCGTGC (SEQ. ID. NO. 3) including start codon and BamHI restriction enzyme site of the alpha and beta forms, forward primer beta form including zebrafish 5′ UTR site: ACTTTCCAAAGCAGACAACCCTCGGG (SEQ. ID. No. 4), reverse primer alpha form including stop codon and EcoRI restriction enzyme site: AAGAATTCTCATGACACTGTGACAACCTCTGTGGAG (SEQ. ID. NO. 5) and reverse primer beta form including 3′ DTR site: AGACATGATTTTGTATCCATACTATTAACAGCTTG (SEQ. ID. NO. 6), cloned alpha form to pcDNA3.1 myc-his vector and beta form to T-easy vector.

Using the cDNA obtained from zebrafish as template, PCR was performed using TAKARA Ex Tag polymerase (TAKARA Company). The cDNA was obtained by extracting RNA from the adult zebrafish and then performing RT-PCR. The RNA was extracted with RNA extraction using TRIZOL and chloroform, and MMLV RT enzyme (BEAMS Company) was used for the RT-PCR. In PCR, in total volume of 50 ul, 2 ul of zebrafish cDNA, 1 ul of the forward and reverse primers, respectively, 0.3 ul of TAKARA Ex Tag polymerase, 5 ul of 10× buffer, and 6 ul of 2.5 mN dNTP were mixed for reaction. The reaction condition included 95° C., 3 minutes (1 time), 95° C. 45 seconds-> 55° C., 45 seconds->72° C., 5 minutes (25 times), 72° C., 10 minutes->maintained at 4° C. (1 time).

### Example 3

### Preparation of Morpholino for Zebrafish AKAP12 (Alpha and Beta Forms) Knockdown

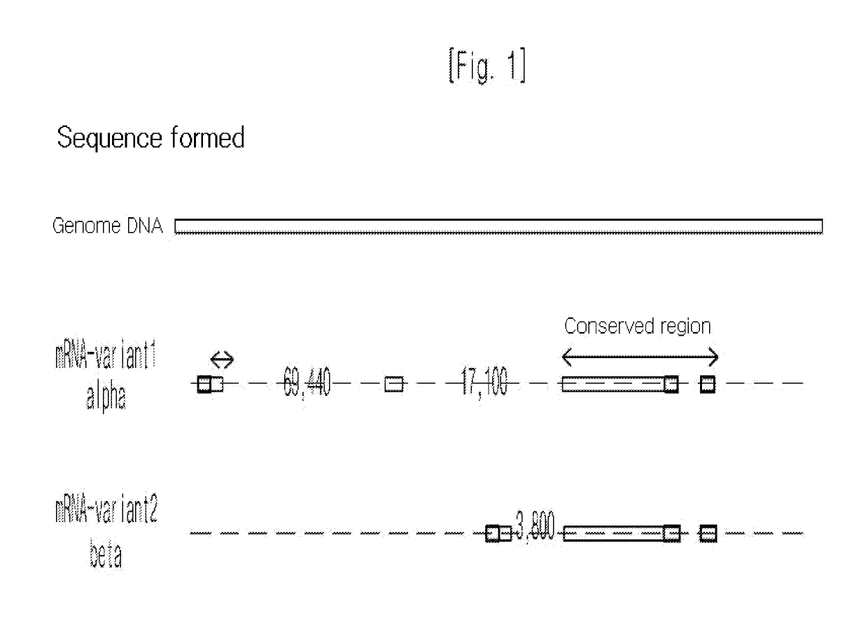

The present inventors prepared morpholino for the zebrafish AKAP12 alpha form mRNA and zebrafish AKAP12 beta form mRNA knockdown. The preparation of the morpholino was ordered to Gene Tools LLC. The method for morpholino preparation is written in Summerton, J. et al., Anti sense & Nucleic Acid Drug Development 7: 187-95, 1997. The prepared morpholino was so prepared to block the characteristic variant regions of each of the alpha and beta forms from binding to the site of splicing and thus block splicing and developing into mature mRNA, in which the alpha form morpholino was so designed to bind adjacent to 339^{th} base of the AKAP12 alpha form as the sequence of TCTTACCTGTTAGAGTTATTGTCCC (SEQ. ID. NO. 7) 25-mer, and the beta form morpholino was so designed to bind adjacent to the 227^{th} base of the AKAP12 beta form as the sequence of TACCTTGCCATCTGCGGTTTCTCCA (SEQ. ID. NO. 8) 25-mer (see FIG. 1).

<Experiment 1> Expression Pattern of Zebrafish AKAP12 Alpha and Beta Forms

To observe the mRNA expression pattern of the zebrafish AKAP12, the present Inventors prepared riboprobe, which corresponded to a base; sequence commonly included in two AKAP12 alpha and beta isoforms, and performed in situ hybridization (ISH).

To be specific, the inventors amplified the common 2258 bp of the AKAP12 alpha and beta isoforms using PCR. The applied base sequence and PCR condition are explained below. For PCR, in total 50 ul of volume, 1 ul of template (AKAP12a/pGEM-T easy vector), 1 ul of forward primer: GAAGAATCTGGTGAACATGTTGTAGGGGAA (SEQ. ID. NO. 9) and 1 ul of reverse primer: GCGACAACCTCAACCTCATTCACTGC (SEQ. ID. NO. 10), respectively, 0.3 ul of TAKARA Ex Tag polymerase, 5 ul of 10× buffer, and 6 ul of 2.5 mM dNTP were mixed with each other for reaction. The reaction condition included 94° C., 3 minutes (1 time), 94° C., 45 seconds->55° C., 45 seconds->72° C., 2 minutes (25 times), 72° C., 10 minutes->maintained at 4° C. (1 time).

After that, the inventors cloned the amplified base sequence to pGEM-T easy vector (Promega). The cloned vector was made linear in forward and backward orientations using two restriction enzymes of SacII and SalI, respectively, and using the linear vectors, the sense and antisense riboprobes regarding the DIG (digoxigenin)-labeled AKAP12 were prepared by in vitro transcription. The applied reaction condition for in vitro transcription will be explained below. In total 20 ul, 1 ug of linear vector, 2 ul of RNAse-free water, 2 ul of 10× DIG-labeled NTP mix, 2 ul of 10× RNA polymerase buffer (Roche), 0.5 ul of RNAse inhibitor, 2 ul of RNA polymerase regarding T7 or SP6 promoter were mixed with each other and reacted for 2 hours at 37° C.

The ISH was performed on the 24, 48 and 72 hour-bid zebrafish embryos after the fertilization using the prepared riboprobes regarding the AKAP12. First, the 24, 48 and 72 hour-old zebrafish embryos were fixed with 4% paraformaldehyde, dehydrated using 100% methanol, and rehydrated with phosphate-buffered saline. After that, the embryos were treated with 10 mg/ml proteinase K, and treated with the above-explained DIG-riboprobes regarding AKAP12 at 65° C. overnight. On the next day, the embryos were rinsed with saline sodium citrate and 0.2× saline sodium citrate, underwent blocking, and treated with anti-DIG-alkaline phosphatase. Next, the embryos were dyed with nitro blue tetrazolium/5-bromo-4-chloro-3-indoyl phosphate p-toluidine (Roche, mixed), and stored in 75% glycerol. The base sequence of the riboprobe is represented by SEQ. ID. NO. 11, and this is a sense base sequence of AKAP12 corresponding to riboprobe. Antisense RNA sequence regarding a base sequence represented by SEQ. ID. NO. 11 was used for the AKAP12 riboprobe used in the above-mentioned experiment.

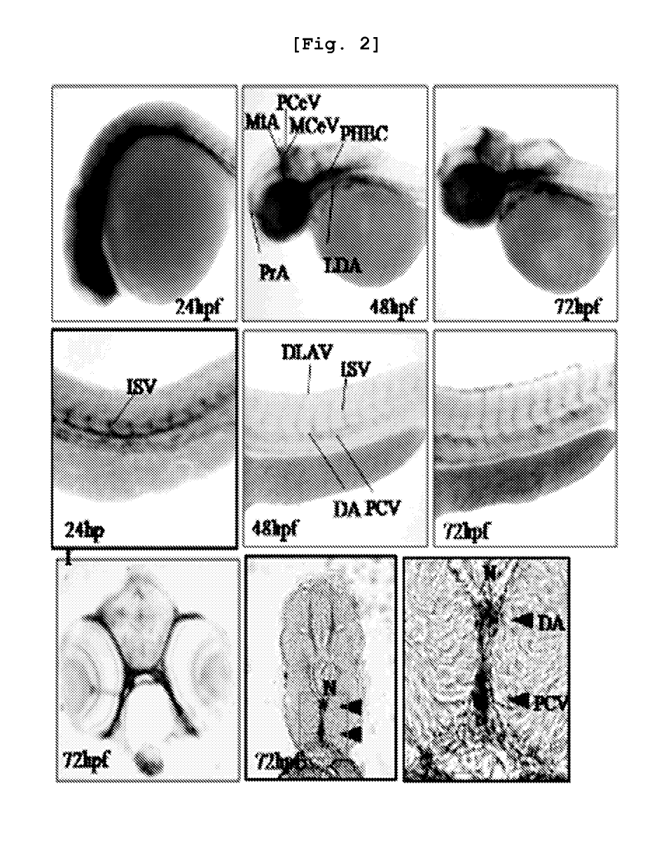

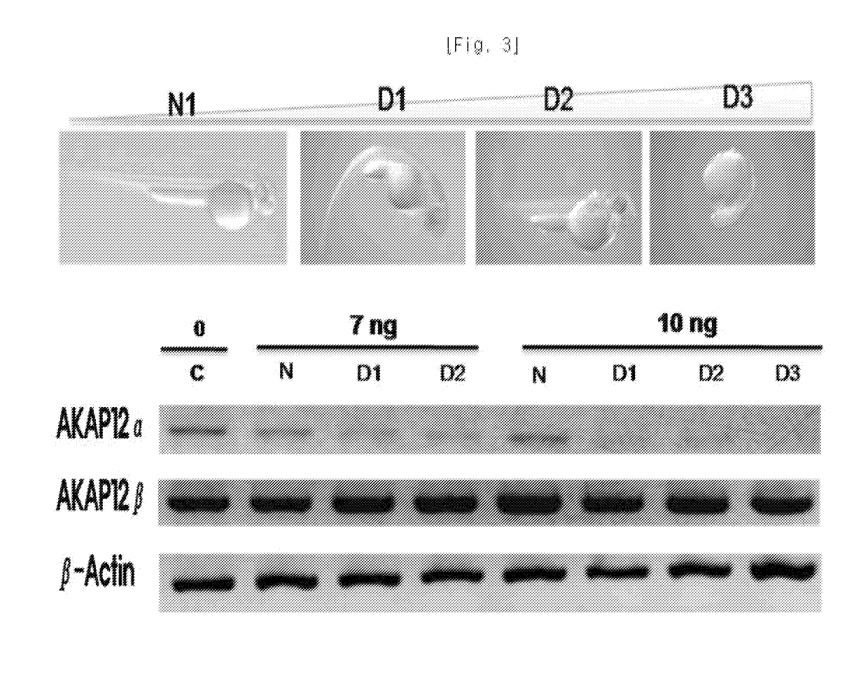

As a result of the ISH, The AKAP12 was expressed generally in blastoderm until 24 hours after the fertilization, but the expression was limited along the head and large vessels (dorsal aorta, DA), posterior cardinal vein (PCV), and intersegmental vessels (ISV), after 24 hours (see FIG. 2).

<Experiment 2> Study on Genetic Defect of AKAP12 Alpha and Beta Form Knockdown Zebrafish

The present invention knocked down zebrafish AKAP12 mRNA by microinjecting the prepared morpholino of <Example 3> into the zebrafish and observed the phenotype.

First, the inventors microinjected 7 ng and 10 ng of morpholino for AKAP12 mRNA alpha form, and microinjected 7.5 ng of morpholino for AKAP12 mRNA beta form.

As a result, the inventors observed that the normal zebrafish without the zebrafish AKAP12 mRNA knockdown showed straightforward and linear tail portions and normal mobility function, whereas the AKAP12 mRNA alpha and beta form knockdown zebrafish showed crooked or shortened tail portions and abnormal mobility.

Further, the present inventors also investigated mRNA expression patterns of the AKAP12 alpha form by RT-PCR in the AKAP12 alpha form knockdown zebrafish into which 7 ng and 10 ng of morpholino for zebrafish AKAP12 alpha form was injected, and could confirm that the zebrafish with severer degree of defects had lower degree of mRNA expression (see FIG. 3).

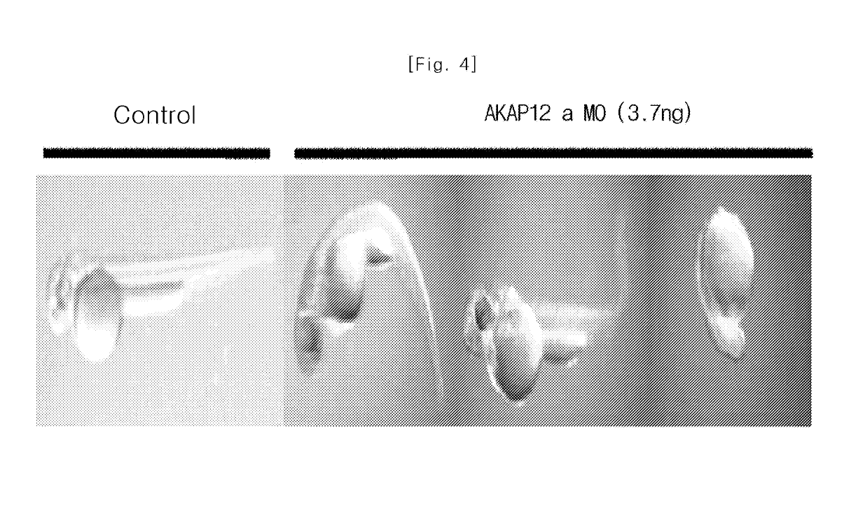

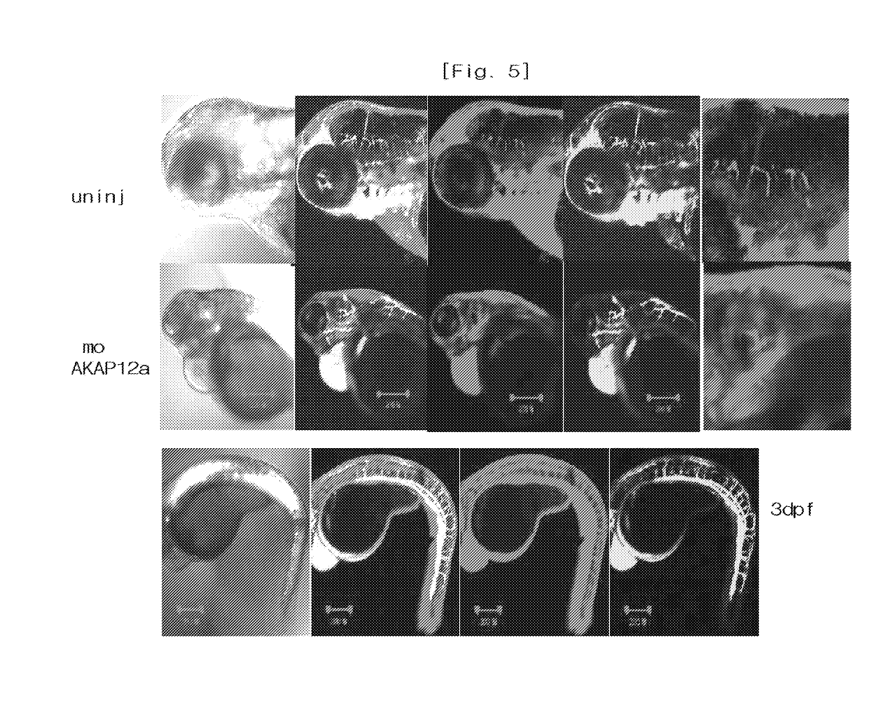

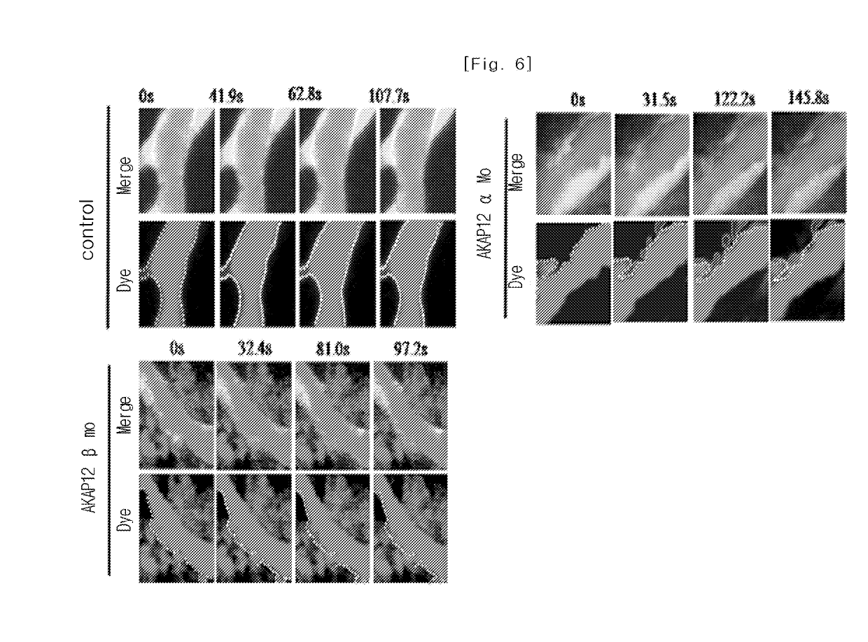

Further, after injecting 3.7 ng, 7.5 ng and 10 ng of morpholino for zebrafish AKAP12 alpha and beta forms, the present inventors could confirm that the genetic defect appears in the beta form at about 7.5 ng and alpha form at about 3.7 ng (see FIG. 4).

<Experiment 3> Study on Micro-Vasculature Defect in Brains of the AKAP12 Alpha Form Knockdown Zebrafish

The present inventor knocked down the zebrafish AKAP12 alpha form using the transgenic zebrafish ‘tg(fli:egfp)’ in which green fluoresces selectively in the vein endothelial cells, and observed the defect pattern of the micro-vasculatures in brains. To be specific, the inventors knocked down the zebrafish AKAP12 alpha form by microinjecting 3 ng of morpholino for zebrafish AKAP12 alpha form into the transgenic zebrafish (fli:egfp) embryos, and then after 3 days, microinjected 25 mg/ml of red-fluorescent lysine-fixable tetramethylrhodamine-dextran, 10 kDa through the common cardinal vein of the zebrafish, and then observed through the LSM 510 META NLO confocal microscope (Carl Zeiss, Germany).

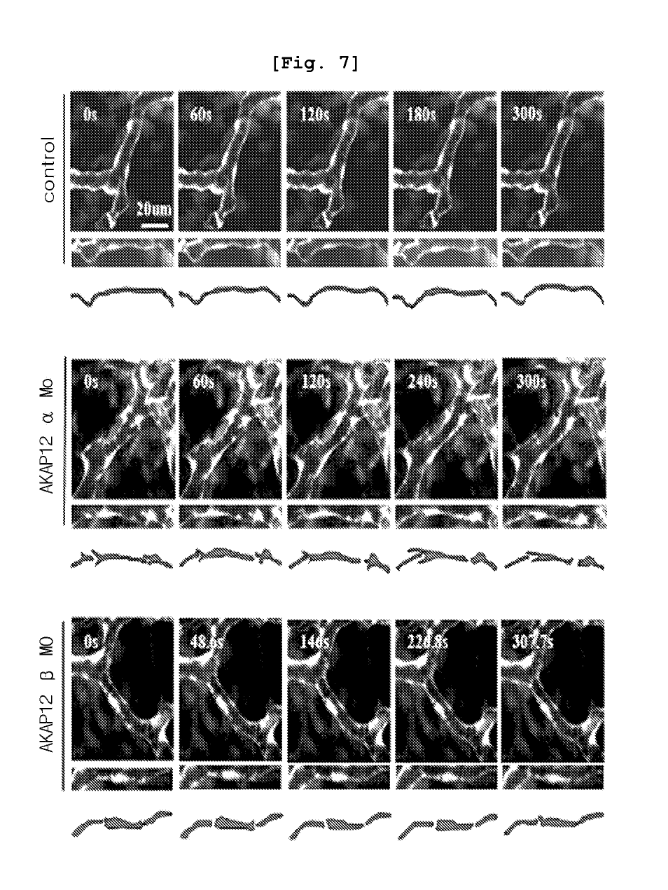

As a result, the inventors could observe that the red lysine-fixable tetramethylrhodamine-dextran stayed within the micro-vasculature in the brains of the normal zebrafish into which morpholino had not been injected, whereas the zebrafish injected with morpholino for AKAP12 alpha form showed non-uniform micro-vasculature in brains and also the red lysine-fixable tetramethylrhodamine-dextran was distributed around the green micro-vasculatures (see FIG. 7).

<Experiment 4> Study on Vessel Detects of AKAP12 Alpha and Beta Form Knockdown Zebrafish

The present inventors knocked down the zebrafish AKAP12 alpha and beta forms using the transgenic zebrafish ‘tg(fli:egfp)’ in which green fluoresces selectively in the endothelioyte of the vessels, and observed the defect pattern in the vessels. To be specific, the inventors knocked down the zebrafish AKAP12 alpha and beta forms by microinjecting 2 ng alpha) and 7.5 ng (beta) of morpholino for zebrafish KAAP12 alpha and beta forms, into the transgenic zebrafish ‘fli:egfp’, respectively, and within 48 to 60 hours, microinjected 25 mg/ml of red-fluorescent lysine-fixable tetramethylrhodamine-dextran, 2000 kDa (molecular probes) and observed through the LSM 510 META NLO confocal microscope (Carl Zeiss, Germany).

As a result, the inventors could observe that red lysine-fixable tetramethylrhodamine-dextran stayed within the vessel of the normal zebrafish which had not been injected with morpholino, whereas the zebrafish injected with morpholino for AKAP12-alpha form showed non-uniform micro-vasculature in the brains and also the red lysine-fixable tetramethylrhodamine-dextran leaked out of the green vessel (see FIG. 6).

<Experiment 5> Study on the Movement of Vein Endothelial Cells of the AKAP12 Alpha and Beta Form Knockdown Zebrafish

The present inventors observed the endotheliocyte of the vessels adjacent to a site of vessel defect with the method explained above in <Experiment 4> using the transgenic zebrafish ‘fli:egfp’, and as a result, could observe that the vein endothelial cells in the AKAP12 alpha and beta form knockdown zebrafish showed more loose contacts between the cells and more active movements compared to the normal vessels (see FIG. 7).

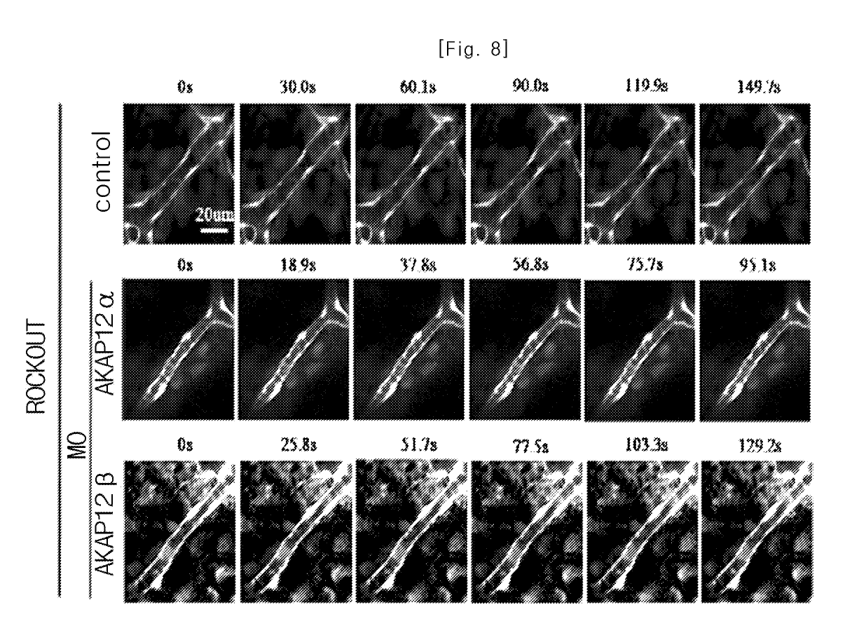

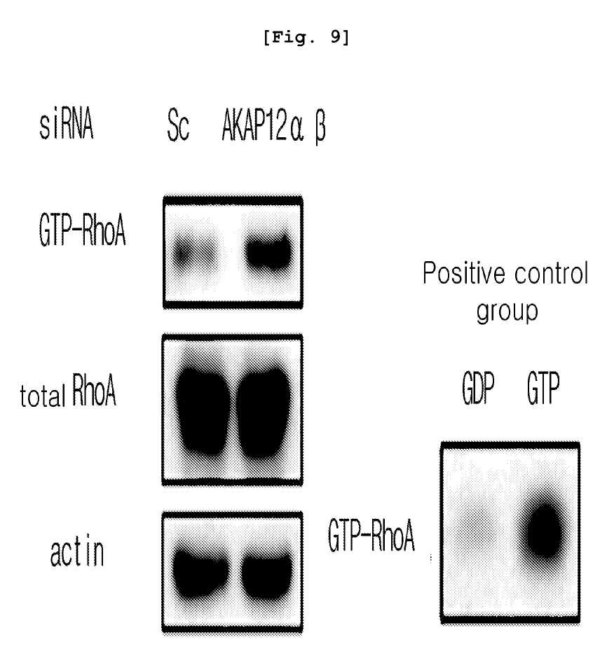

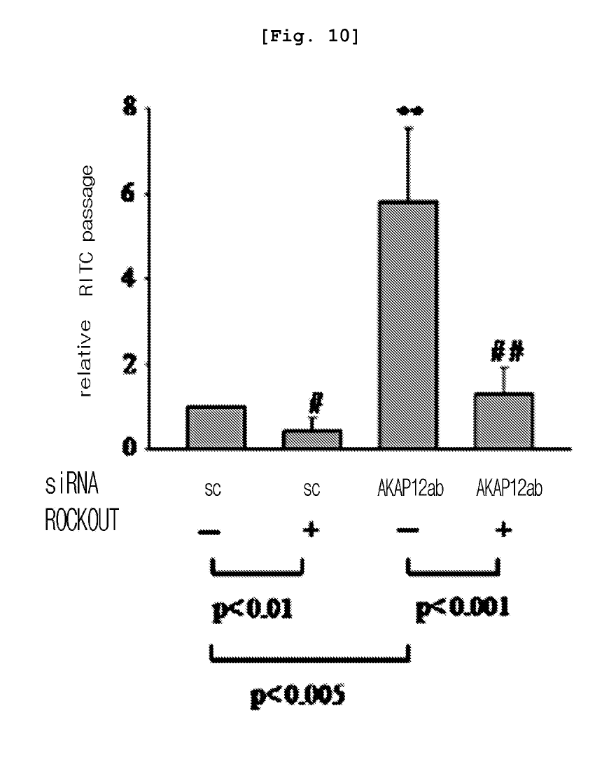

Further, in order to verify a link between the active movement of the vein endothelial cells of the AKAP12 knockdown zebrafish with RhoA, which is one of the small GTPase proteins, 44 hours after the fertilization of the AKAP12 knockdown zebrafish, the inventors treated with 200 uM of ROCKOUT (CALBIOCHEM) as a RhoA signal inhibitor, and as a result, could observe the excessive movement of the vein endothelial cells slowed (see FIG. 8).

Further, using the collagenase, and using Human Umbilical Vein Endothelial Cells (HUVEC) isolated from the human umbilical cord vein (Catholic University of Korea, School of Medicine), the inventors confirmed the above result in vitro. To be specific, the inventors knocked down APAP12 in HUVEC using siRNA which can knock down both the AKAP12 alpha and beta concurrently in a cell strain, and analyzed with the RhoA-GTP binding assay, to confirm that increase of GTP-RhoA which is the active form of the RhoA in the AKAP12 knockdown HUVEC (see FIG. 9).

Further, as a result of performing the permeability assay in vitro using HUVEC (the permeability assay was measured based on the degree of inter-cell permeation by using type I collagen-coated transwell units (6.5 mm diameter, 3.0 m pore size polycarbonate filter; Costar, Cambridge, Mass.) by treating the upper chamber with 0.1 mg/ml Rhodamine β isothiocyanate (RITC)-labeled dextran (molecular weight, 10,000). When treated with ROCKOUT, incubation was performed for 15 minutes after the drug treatment, and then the degree of fluorescence was measured, from the lower compartment diluted with 50 ml PBS using the spectrophotometer (Spectra MAX Gemini XS; Molecular device, USA), the inventors could confirm that a group (AKAP12ab) treated with AKAP12 siRNA had increased RITC permeability than a control siRNA (sc), and decreased permeability when treated with ROCKOUT (see FIG. 10).

Further, in order to verify the link between the loosened contact between the vein endothelial cells of the AKAP12 knockdown zebrafish and the defect in the cell-cell adhesion proteins, the inventors confirmed by the western-blotting and immunocytochemistry that, when the AKAP12 gene was knocked down using siRNA, expression of one of the adhesion proteins, i.e., ve-cadherin was down-regulated in the cell membrane.

To be specific, in the western-blotting, first, cells were dissolved in lysis buffer containing 10 mM HEPES (pH 7.9), 400 mM NaCl, 0.1 mM EDTA, 5% glycerol, 1 mM DTT (D1-dithiothreitol) and protease inhibitor cocktail, and the proteins in cells were quantified using BCA. From the whole cell lysate prepared as explained above, the proteins in cells were isolated according to molecular weight sizes using sodium dodecyl sulfate-polyacrylamide gel electrophoresis, and the proteins were moved to the polyvinyldifluoride membrane (Millipore). After that, blocking was performed, and the primary antibody was treated overnight (for 16 hours) at 4° C. On the next day, secondary antibodies regarding the primary antibodies were treated at room temperature for 2 hours, respectively, and detected using ECL PLUS (Amersham). As for the primary antibodies, VE-cadherin (Santa Cruz Biotechnology); F-actin (Sigma) were used.

As for the immunochemistry, first, cells were seeded in a 0.1% gelatin-coated slide, rinsed with cold PBS, and fixed for 10 minutes at room temperature with 4% paraformaldehyde. After that, the cells were rinsed again three times with PBS, treated with 0.5% Triton X-100 for 5 minutes, rinsed with PBS-T and underwent blocking at room temperature for 30 minutes with 0.1% blocking solution (Roche). After blocking, the primary antibodies were treated at 4° C. for 16 hours, the second fluorescent antibodies regarding the primary antibodies were treated at room temperature for 2 hours, respectively, rinsed, DAPI dyed, and mounted and the cells were observed through Zeiss LSMS10 meta MLO confocal microscope (Zeiss, Obserkochen, Germany; KBSI, Chuncheon Center). VE-cadherin ((Santa Crus Biotechnology); F-actin (Sigma) were used for the primary antibodies.

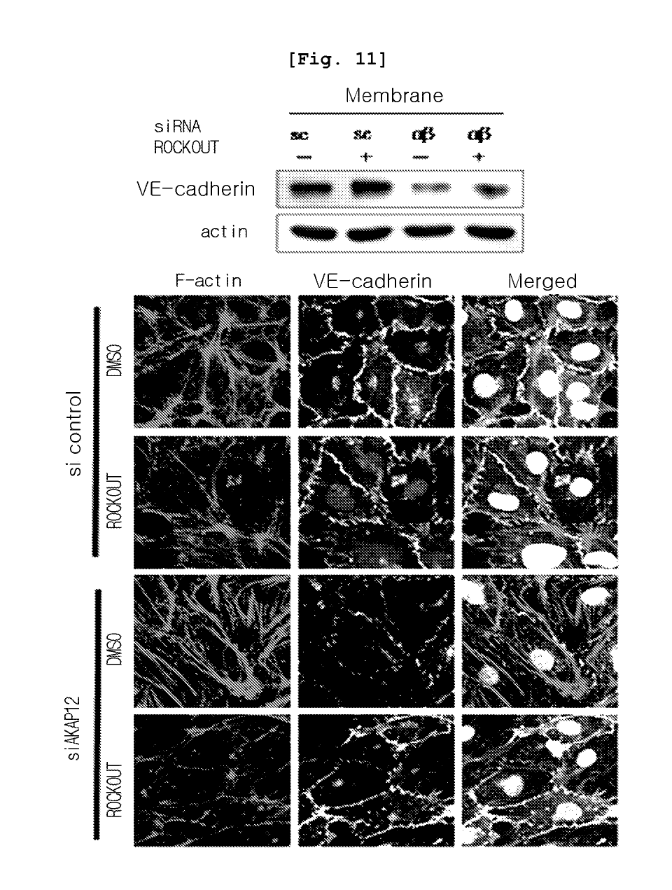

As a result, the inventors could confirm that when treated with both siRNA regarding AKAP12 and ROCKOUT, the expression of ve-cadherin increased back (see FIG. 11).

<Experiment 6> Study on Heart Defect of the AKAP Alpha and Beta Form Knockdown Zebrafish

The present inventors knocked down zebrafish AKAP12 alpha form using the transgenic zebrafish ‘tg(cmlc:egfp)’ in which green fluoresces characteristically to the light chain of the heat muscles, and observed the heart defects of the zebrafish. That is, the inventors knocked down the zebrafish AKAP12 alpha form by microinjecting 3.7 ng of morpholino for zebrafish AKAP12 alpha form into the transgenic zebrafish (‘tg(cmlc:egfp)’) embryos, and observed the hearts of the zebrafish after 1.5 days later using the LSM 510 META NLO confocal microscope Carl Zeiss, Germany).

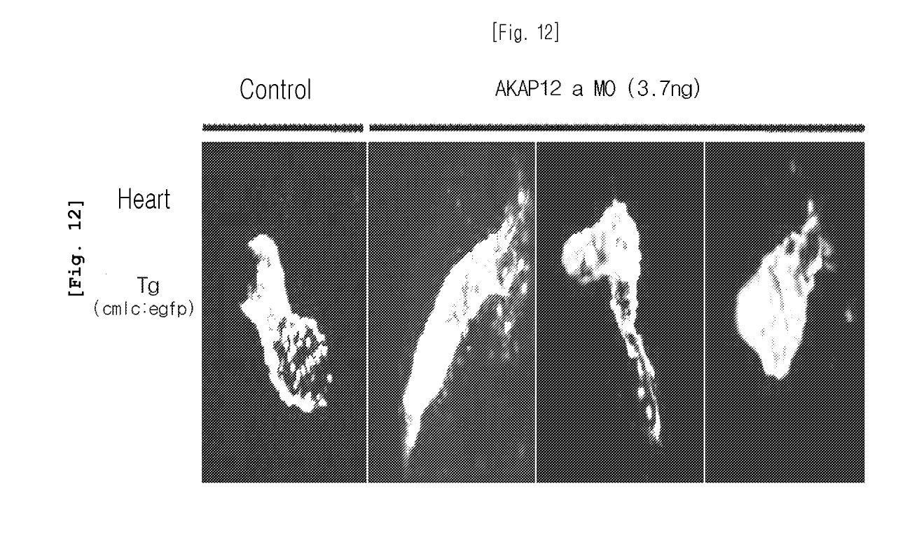

As a result, the inventors could observe that the zebrafish injected with morpholino for AKAP12 alpha form showed elongated atriums and ventricles and additionally, unlike the normal zebrafish heart having the atrium on a left side and the ventricle on the right, the atriums and the ventricles were placed on a line. Further, instead of constant heartbeats, the zebrafish injected with morpholino for AKAP12 alpha form had non-uniform and weak heartbeats (see FIG. 12). The blood circulation was also more inefficient as the defects were severer.

<Experiment 7> Study on Hemorrhage of AKAP Alpha and Beta Form Knockdown Zebrafish

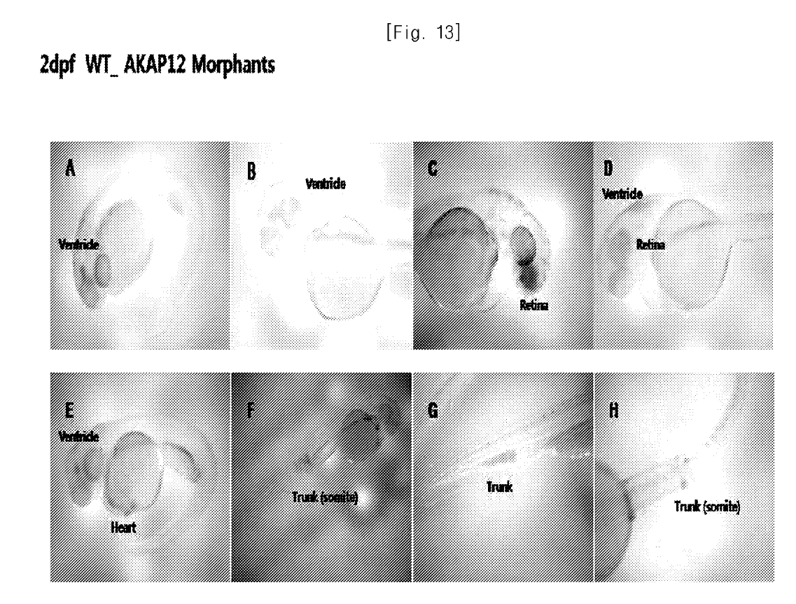

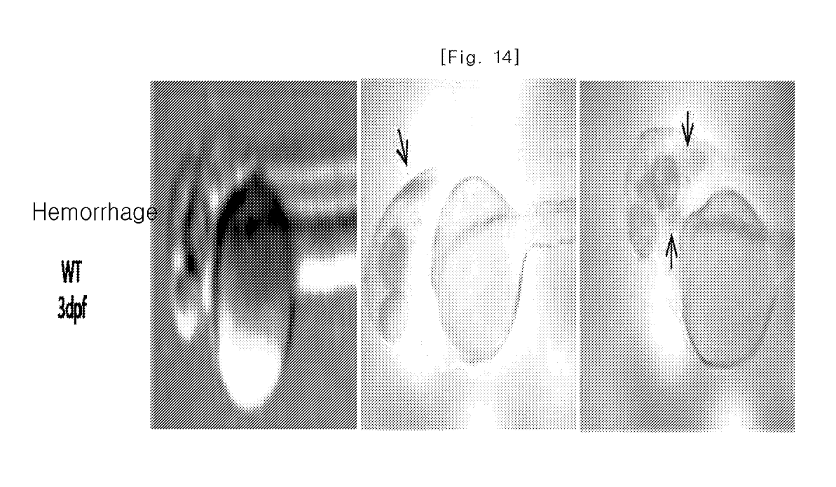

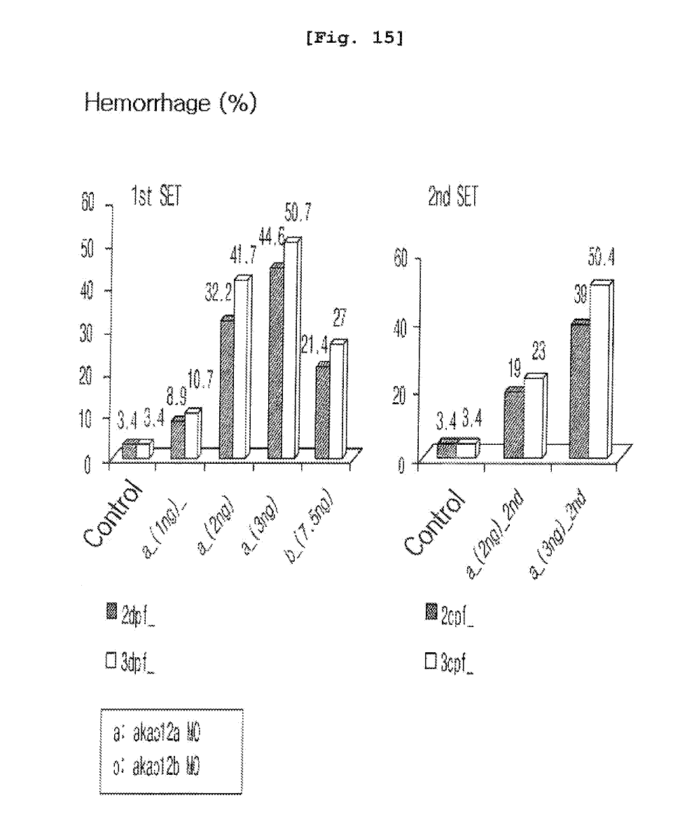

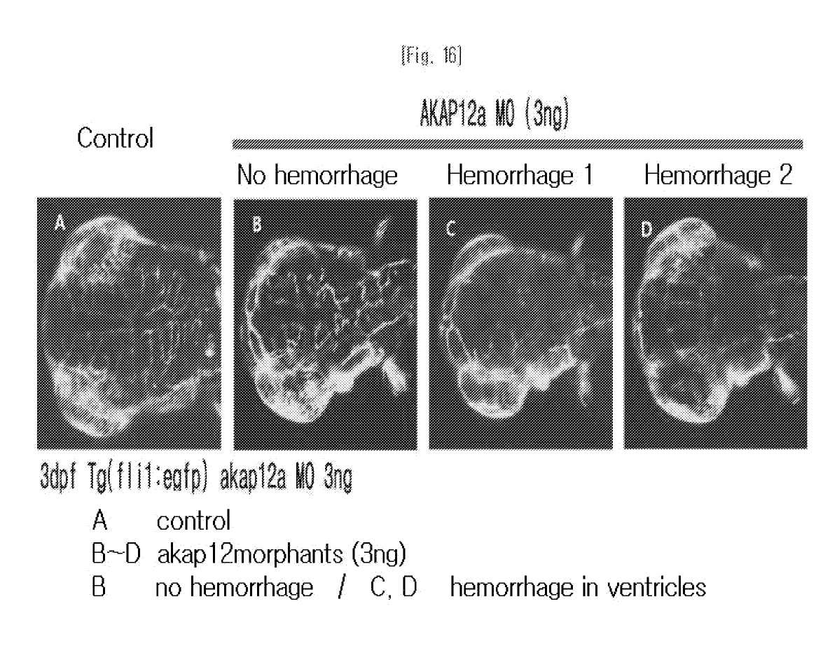

The present inventors observed the embryos of adult AKAP12 alpha and beta form knockdown zebrafish, and as a result, could observe the bleeding in the 2 to 3 day-old zebrafish. The bleeding was generally observed from the ventricles of the brains, retinas, hearts and trunks, and from statistics obtained by injecting morpholino for zebrafish AKAP12 alpha form by 1, 2 and 3 ng in sequence, the rate of zebrafish with, hemorrhage increased as the amount of injected morpholino increased. Further, the increased rate of zebrafish with hemorrhage was observed at day 3, based on the observation made 2 to 3 days after injecting morpholino for zebrafish AKAP12 alpha and beta forms. With reference to the observation at day 3, approximately 50% of zebrafish showed hemorrhage when 3 ng of morpholino for zebrafish AKAP12 alpha form was injected, and approximately 27% of zebrafish showed hemorrhage when 7.5 ng of morpholino for zebrafish AKAP12 beta form was injected (see FIGS. 13, 14 and 15).

The present inventors observed the vasculature pattern in brains of the zebrafish with the hemorrhage observed as explained above. In order to observe the vasculature pattern, the inventors microinjected morpholino for zebrafish AKAP12 alpha form into the transgenic (tg(fli:egfp)) zebrafish embryos, divided the zebrafish into two groups with and without hemorrhage, and observed the vasculatures in brains through the LSM 510 META NLO confocal microscope (Carl Zeiss, Germany).

As a result, the group without hemorrhage formed veins, although these showed non-uniform vasculature patterns with curves, which is different from the brain vein of the normal zebrafish. However, the group with hemorrhage formed thinned or regressed brain veins along with the non-uniform vasculature patterns in the brains (see FIG. 16).

<Experiment 8> Study on Zebrafish Defects in Accordance with Morpholino Concentration

The present inventors knocked down AKAP12 alpha form mRNA and beta form mRNA using morpholino for zebrafish AKAP12 alpha and beta forms, and observed the defect patterns in accordance with the amounts of morpholino, respectively.

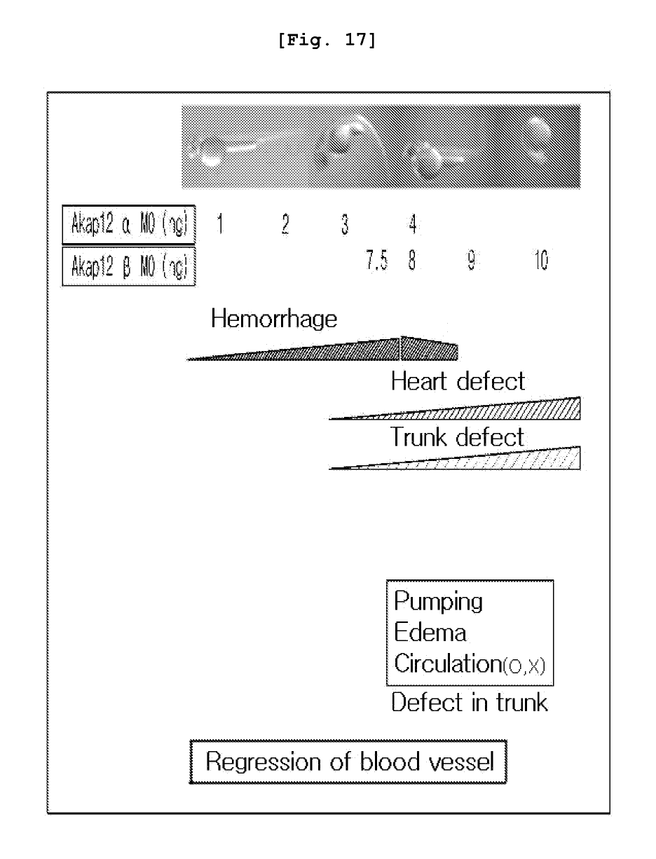

As a result, the zebrafish treated with 1 ng and 2 ng of morpholino for zebrafish AKAP12 alpha form showed bleeding only, but those treated with 3 and 4 ng of morpholino showed both the bleeding and defects in heart and trunk veins. The zebrafish treated with 4 ng and more of morpholino showed decreasing bleeding due to severe defects of hearts and other veins and subsequently, showed inefficient blood circulation. Further, when treated with 7.5 ng and 8 ng of morpholino for zebrafish AKAP12 beta form, the zebrafish showed both the bleeding and defects of heat and trunk veins concurrently, and those treated with 9 ng or more of morpholino showed only the defects in heart and trunk veins (see FIG. 17).

<Experiment 9> Rescue Test by AKAP12 Alpha and Beta Form mRNA of the AKAP12 Alpha and Beta Form Knockdown Zebrafish

In order to verify the link between the defects in the shapes and hemorrhage defects with the AKAP12-specific knockdown, the present inventors conducted the rescue experiment using zebrafish AKAP12 alpha and beta form mRNA. To be specific, the inventors microinjected morpholino for zebrafish AKAP12 alpha and beta forms and mRNA of the zebrafish AKAP12 alpha and beta forms into the zebrafish embryos, and confirmed changes in the development and bleeding as explained in <Experiment 2> and <Experiment 7>.

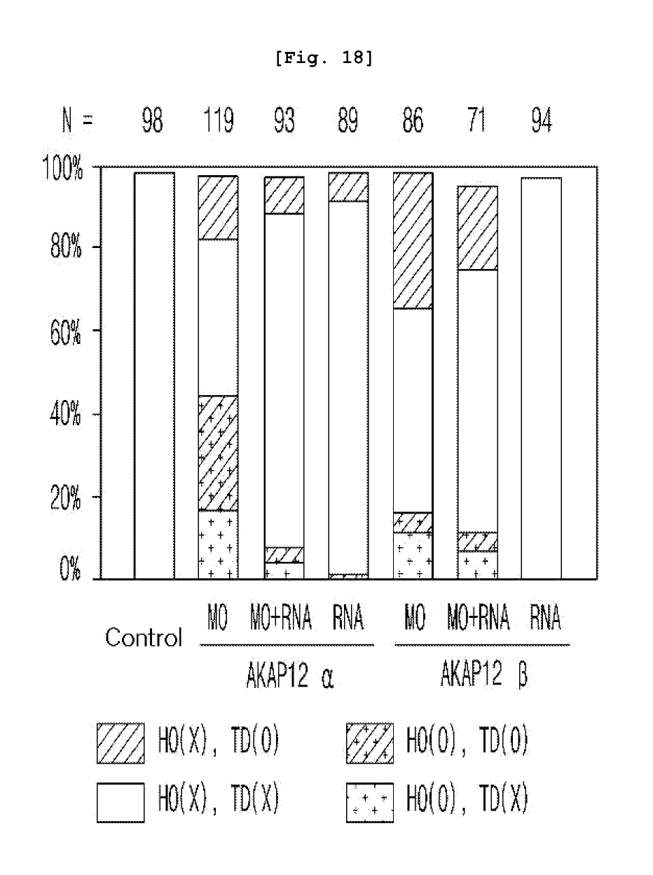

As a result, the inventors could confirm that a control group microinjected with morpholino for AKAP12 alpha and beta forms only showed crooked trunks and bleeding, defect patterns, but the rate of zebrafish with improved trunk defects and bleeding pattern increased among the zebrafish microinjected with the zebrafish AKAP12 alpha and beta form mRNA and morpholino (see FIG. 18).

<Experiment 10> Rescue Experiment by Rat AKAP12 Alpha Form mRNA of the AKAP12 Alpha and Beta Form Knockdown Zebrafish

In order to verify the link between the defects observed in <Experiment 1> to <Experiment 5> and the AKAP12-specific knockdown, the present inventors conducted rescue experiment using rat AKAP12 mRNA. That is, the inventors mixed 4 ng of morpholino for zebrafish AKAP12 alpha form and 50 pg and 100 pg of rat AKAP12 alpha form, and microinjected the mixture into the zebrafish embryos and observed.

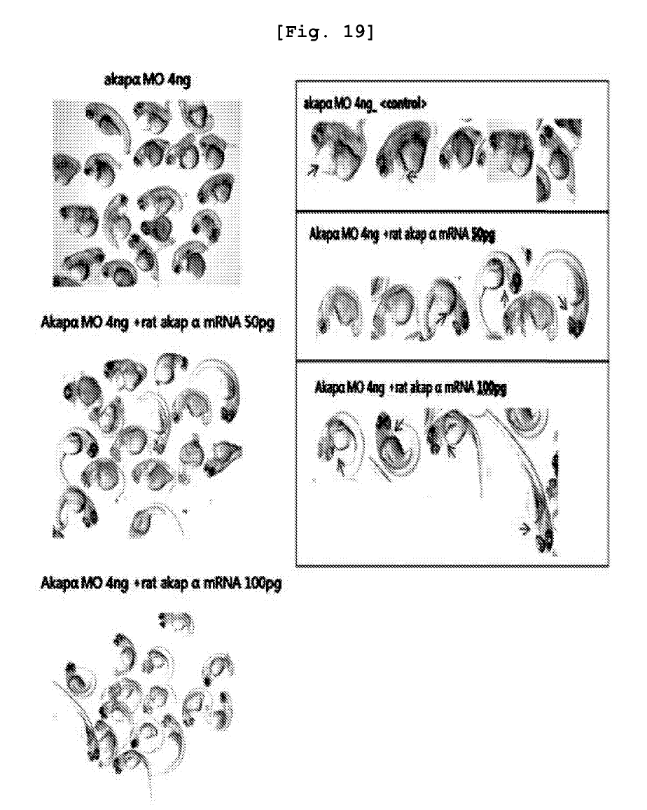

As a result, the inventors could confirm that a control group injected with only 4 ng of morpholino for AKAP12 alpha form showed defect patterns including crooked and shortened tail and deformed hearts, whereas the experiment group microinjected with the mixture of rat AKAP12 alpha form mRNA and morpholino showed increased rate of zebrafish with improved defect patterns in tails and hearts as the amount of mRNA increased (see FIG. 19).

### INDUSTRIAL APPLICABILITY

As explained above, AKAP12 can be effectively used as a composition for prevention and treatment of a circulatory-defect, a medicine for prevention and treatment of a genetic defect, and a hemorrhage inhibitor, and the AKAP12-deficient mutant zebrafish can be effectively used as an animal model for screening a medicine for prevention and treatment of a circulatory or a genetic defect.

1. A composition comprised of AKAP12 (A-Kinase anchoring protein 12) as an active component, for prevention and treatment of a circulatory defect induced due to AKAP12 deficiency.

2. The composition of claim 1, wherein the AKAP12 is an AKAP12 alpha form or an AKAP12 beta form.

3. The composition of claim 2, wherein the AKAP12 alpha form is a nucleic sequence represented by SEQ. ID. NO. 1, and the AKAP12 beta form is a nucleic sequence represented by SEQ. ID. NO. 2.

4. The composition of claim 1, wherein the circulatory defect is at least one selected from a group consisting of a micro-vasculature defect in brain, a defect in heart, and a defect in a vein.

5. A composition comprised of AKAP12 (A-Kinase anchoring protein 12) as an active component, for prevention and treatment of a genetic defect induced due to AKAP12 deficiency.

6. A composition comprised of AKAP12 (A-Kinase anchoring protein 12) as an active component for hemorrhage inhibition.

7. A method for preventing or treating a circulatory defect, comprising the step of administering a pharmaceutically-effective amount of the composition of claim 1.

8. A method for preventing or treating a genetic defect, comprising the step of administering a pharmaceutically-effective amount of the composition of claim 1.

9. A method for inhibiting hemorrhage, comprising the step of administering a pharmaceutically-effective amount of the composition of claim 1.

10-12. (canceled)

13. An AKAP12 (A-Kinase anchoring protein 12)-deficient mutant animal having a circulatory or genetic defect.

14. The AKAP12-deficient mutant animal of claim 13, wherein mutation comprises an AKAP12 gene knockout or knockdown.

15. The AKAP12-deficient mutant animal of claim 14, wherein the AKAP12 gene is an AKAP12 alpha form or an AKAP12 beta form.

16. The AKAP12-deficient mutant animal of claim 13, wherein the animal is one selected from a group consisting of zebrafish, mouse, rat, pig and monkey.

17. A method for screening a medicine for prevention and treatment of a circulatory defect, the method comprising the steps of:
1) administering a candidate for prevention and treatment of a circulatory defect into an AKAP12 gene knockout or knockdown animal;
2) confirming a degree of development of a circulatory system of the animal into which the candidate for prevention and treatment of the circulatory defect is administered in step 1); and
3) selecting a candidate which meaningfully recovers the degree of development of the circulatory system, by comparing with a control group animal into which the candidate is not administered.

18. A method for screening a medicine for prevention and treatment of a genetic defect, comprising the steps of:
1) administering a candidate for prevention and treatment of the genetic defect into an AkAP12 gene knockout or knockdown animal;
2) confirming a degree of genetic development of the animal into which the candidate for prevention and treatment of the genetic defect is administered in step 1); and
3) selecting a candidate which meaningfully recovers the degree of genetic developments by comparing with a control group animal into which the candidate is not administered.

19. The method of claim 17, wherein the AKAP12 gene of step 1) is an AKAP12 alpha form or an AKAP12 beta form.

20. The method of claim 17, wherein the animal of step 1) is one selected from a group consisting of zebrafish, mouse, rat, pig and monkey.

21. The method of claim 17, wherein the candidate of step 1) is one selected from a group consisting of peptide, protein, non-peptide compound, synthesized compound, fermented product, cell extract, plant extract, extract from animal tissue and plasma.

22. The method of claim 18, wherein the AKAP12 gene of step 1) is an AKAP12 alpha form or an AKAP12 beta form.

23. The method of claim 18, wherein the animal of step 1) is one selected from a group consisting of zebrafish, mouse, rat, pig and monkey.

24. The method of claim 18, wherein the candidate of step 1) is one selected from a group consisting of peptide, protein, non-peptide compound, synthesized compound, fermented product, cell extract, plant extract, extract from animal tissue and plasma.
